(19) United States
(12) Patent Application Publication
(10) Pub. No.: US 2022/0143242 A1
(43) Pub. Date: May 12, 2022

(54) Chair Sanitizing Assembly

(71) Applicant: Saul Braha, Brooklyn, NY (US)

(72) Inventor: Saul Braha, Brooklyn, NY (US)

(21) Appl. No.: 17/094,331

(22) Filed: Nov. 10, 2020

(51) Int. Cl. A61L 2/18; A61L 2/26

(52) U.S. Cl. CPC A61L 2/18; A61L 2202/18; A61L 2202/15; A61L 2/26

(57) ABSTRACT

A chair sanitizing assembly for disinfecting a chair includes a cylinder that can be attached to a chair and a bottle containing a disinfectant solution that is insertable into the cylinder. A first hose is fluidly coupled to the bottle to receive the disinfectant solution. The first hose extends into a puck and a pump is positioned within the puck to pump the disinfectant solution. A second hose is fluidly coupled to the pump to receive the disinfectant solution when the pump is turned on. A spray nozzle is coupled to the second hose to spray the disinfectant solution onto the chair portion of the chair thereby disinfecting the chair portion prior the user sitting on the chair portion.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sanitizing devices and more particularly pertains to a new sanitizing device for sanitizing a chair in a public area.

### (2) Description of Related Art Including Information Disclosed Under 37 CFR 1.97 and 1.98

The prior art relates to sanitizing devices including a variety of disinfectant spraying devices that are mountable on or near a toilet seat. The prior art disclose a variety of motion sensing disinfectant sprayers that are mountable over a door knob for disinfecting the door knob when the door knob is manipulated. Additionally, the prior art discloses a variety of overhead disinfectant spraying devices for disinfecting a large, open area.

### BRIEF SUMMARY OF THE INVENTION

An embodiment of the disclosure meets the needs presented above by generally comprising a cylinder that can be attached to a chair and a bottle containing a disinfectant solution that is insertable into the cylinder. A first hose is fluidly coupled to the bottle to receive the disinfectant solution. The first hose extends into a puck and a pump is positioned within the puck to pump the disinfectant solution. A second hose is fluidly coupled to the pump to receive the disinfectant solution when the pump is turned on. A spray nozzle is coupled to the second hose to spray the disinfectant solution onto the chair portion of the chair thereby disinfecting the chair portion prior the user sitting on the chair portion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a chair sanitizing assembly according to an embodiment of the disclosure.

FIG. 2 is a top exploded view of an embodiment of the disclosure.

FIG. 3 is a bottom phantom view of an embodiment of the disclosure.

FIG. 4 is a left side view of an embodiment of the disclosure.

FIG. 5 is a perspective in-use view of an embodiment of the disclosure.

FIG. 6 is a cross sectional view taken along line 6-6 of FIG. 1 of an embodiment of the disclosure.

### DETAILED DESCRIPTION OF THE INVENTION

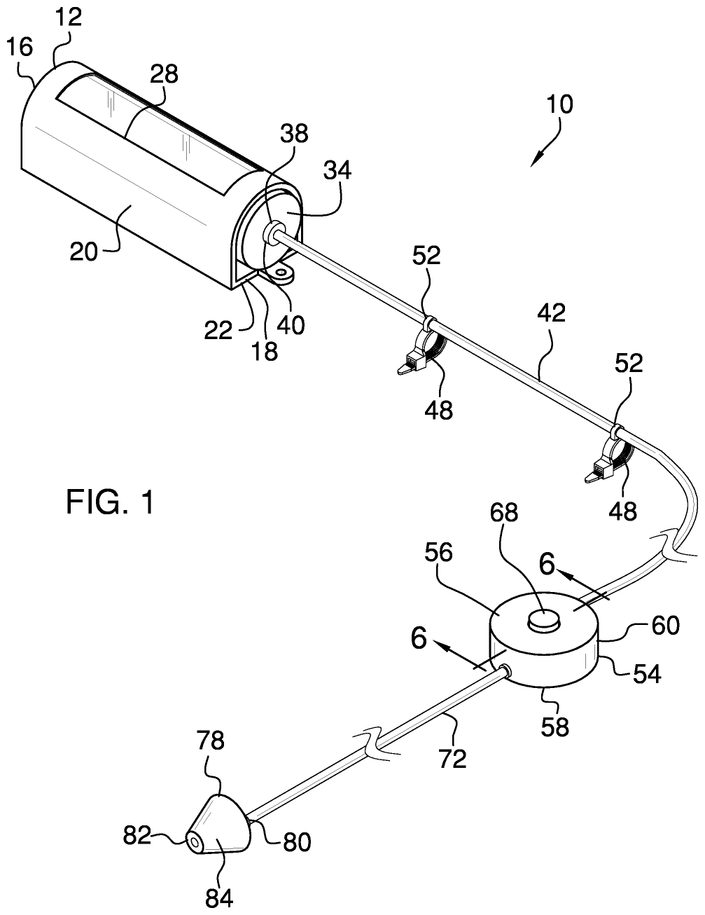

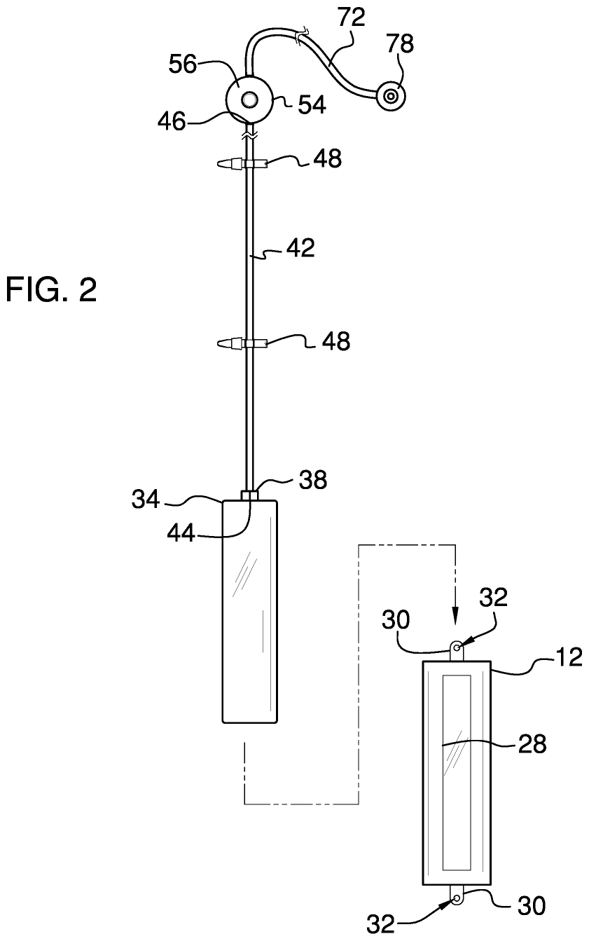

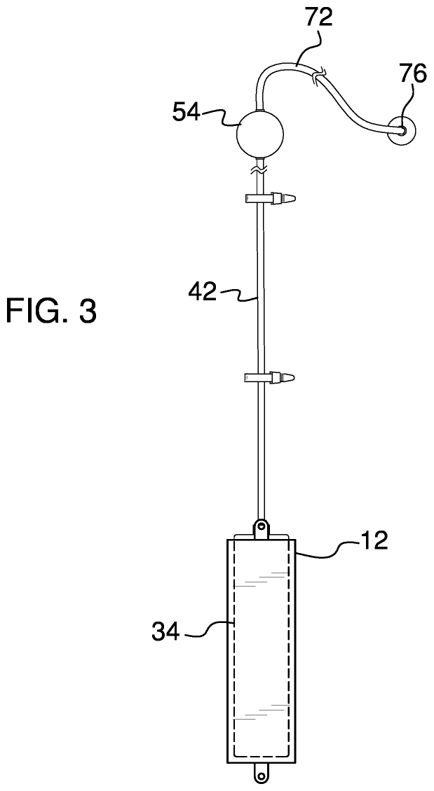

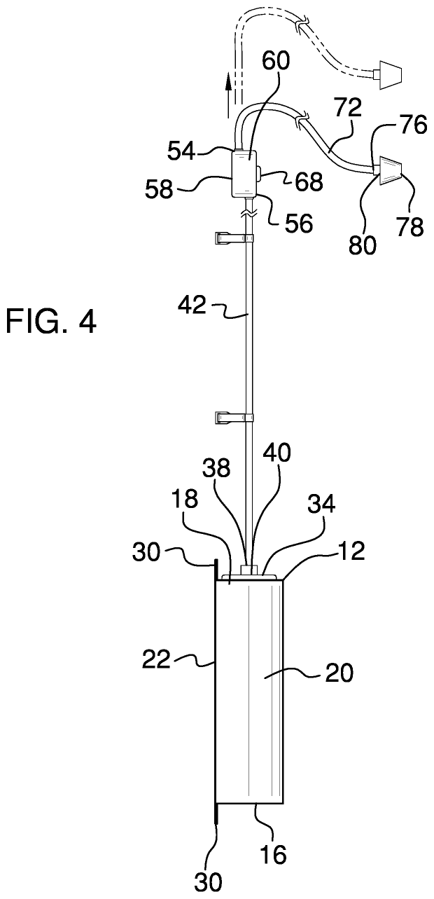

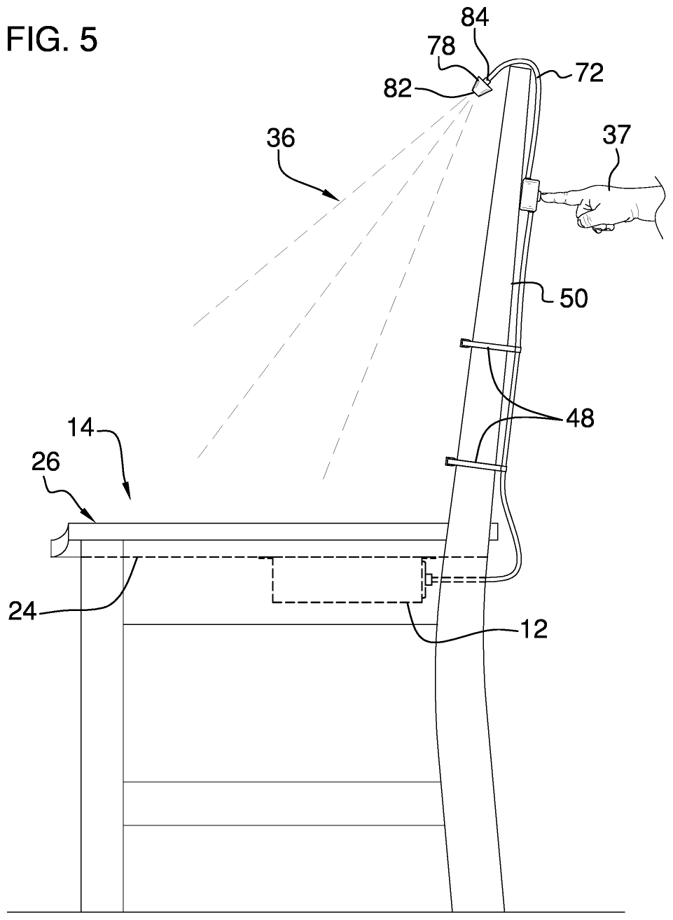

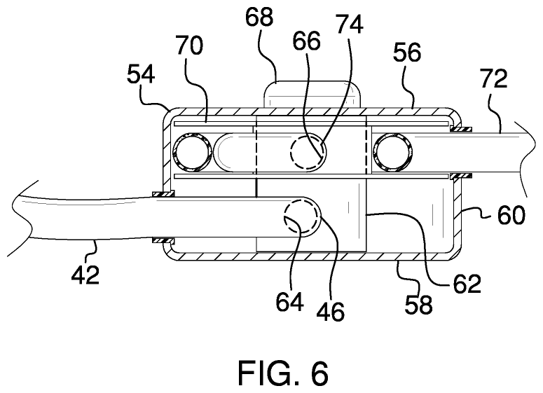

With reference now to the drawings, and in particular to FIGS. 1 through 6 thereof, a new sanitizing device embodying the principles and concepts of an embodiment of the disclosure and generally designated by the reference numeral 10 will be described.

As best illustrated in FIGS. 1 through 6, the chair sanitizing assembly 10 generally comprises a cylinder 12 that can be attached to a chair 14. The chair 14 may be a chair in a stadium, a restaurant or any other public location that would involve multiple users sitting in the chair 14 over time. The cylinder 12 has a first end 16, a second end 18 and an outer wall 20 extending therebetween; the cylinder 12 is hollow and the second end 18 is open. The outer wall 20 has a flattened portion 22 extending between the first end 16 and the second end 18 and the flattened portion 22 lies against a bottom surface 24 of a seat portion 26 of the chair 14.

The outer wall 20 has a window 28 extending therethrough and the window 28 is elongated to extend substantially between the first end 16 and the second end 18. Moreover, the window 28 is positioned on an opposite side of the outer wall 20 with respect to the flattened portion 22. The cylinder 12 has a pair of tabs 30 each extending away from a respective one of the first end 16 and the second end 18 and each of the tabs 30 is aligned with the flattened portion 22. Each of the tabs 30 has a hole 32 extending therethrough to accommodate a fastener, such as a screw or the like, to engage the bottom surface 24 of the seat portion 26 of the chair 14 for attaching the cylinder 12 thereto.

A bottle 34 is included that contains a disinfectant solution 36. The disinfectant solution 36 may be any fluid disinfectant that is approved for use on the human body thereby inhibiting a toxic reaction in a user 37 when the user 37 sits on the chair 14. The bottle 34 is insertable into the cylinder 12 and the bottle 34 has an outlet 38 that is fluidly integrated into a primary end 40 of the bottle 34. The bottle 34 is insertable into the second end 18 of the cylinder 12 having the outlet 38 being exposed. Additionally, the bottle 34 is comprised of a translucent material to facilitate the disinfectant solution 36 to be visible therethrough. In this way the level of the disinfectant solution 36 is visible through the window 28 in the outer wall 20 of the cylinder 12. The bottle 34 is replaced when the disinfectant solution 36 is depleted.

A first hose 42 is provided that is fluidly coupled to the bottle 34 to receive the disinfectant solution 36. The first hose 42 has a first end 44 and a second end 46, and the first end 44 of the first hose 42 is removably and fluidly coupled to the outlet 38 of the bottle 34. A plurality of attachments 48 is provided and the first hose 42 extends through each of the attachments 48. Additionally, each of the attachments 48 engages a backrest 50 of the chair 14 such that the first hose 42 extends upwardly along the backrest 50. Additionally, each of the attachments 48 has a closed loop 52 integrated therein through which the first hose 42 extends. In this way the first hose 42 is not compressed which could potentially inhibit the disinfectant solution 36 from flowing through the first hose 42. Each of the attachments 48 may comprise a zip tie that has a cable loop integrated therein or any other type of releasable fastener that can attach to the backrest 50 of the chair 14 without compressing the first hose 42.

A puck 54 is included and the first hose 42 extends into the puck 54. The puck 54 has a top side 56, a bottom side 58 and a perimeter side 60 extending therethrough. A pump 62 is provided and the pump 62 is positioned within the puck 54. The first hose 42 is fluidly coupled to the pump 62 thereby facilitating the pump 62 to pump the disinfectant solution 36. The pump 62 has an input 64 and an output 66, and the second end 46 of the first hose 42 is fluidly coupled to the input 64. In this way the pump 62 can pump the disinfectant solution 36 outwardly through the output 66 when the pump 62 is turned on. The pump 62 may comprise a manually operated fluid pump that urges a pre-determined volume of fluid each time the pump 62 is manually engaged by the user 37.

A button 68 is movably integrated into the puck 54 and the button 68 can be depressed by the user 37. The button 68 is integrated into the pump 62 and the pump 62 is actuated when the button 68 is depressed to pump a predetermined volume of the disinfectant solution 36. Additionally, the button 68 extends through the top side 56 of the puck 54. A spool 70 is positioned within the puck 54 and the spool 70 surrounds the pump 62. The spool 70 is biased to rotate in a first direction and the spool 70 is urgeable to rotate in a second direction. The spool 70 is aligned with the output 66 of the pump 62 and the spool 70 may comprise a spring loaded spool or the like.

A second hose 72 is fluidly coupled to the pump 62 to receive the disinfectant solution 36 when the pump 62 is turned on. The second hose 72 extends outwardly from the puck 54 and the second hose 72 can be draped over the backrest 50 of the chair 14. The second hose 72 has a first end 74 and a second end 76, and the first end 74 of the second hose 72 is fluidly coupled to the output 66 of the pump 62. The second hose 72 is wrapped around the spool 70 when the spool 70 is biased to rotate in the first direction. Conversely, the spool 70 rotates in the second direction when the second hose 72 is drawn outwardly from the puck 54.

A spray nozzle 78 is coupled to the second hose 72 thereby facilitating the spray nozzle 78 to spray the disinfectant solution 36 onto the chair 14 portion of the chair 14. In this way the chair 14 portion can be disinfected prior the user 37 sitting on the chair 14 portion. Thus, the chair 14 is inhibited from spreading an infectious disease between multiple users. The spray nozzle 78 has a primary end 80, a secondary end 82 and an outside wall 84 extending therebetween. The spray nozzle 78 is hollow, each of the primary end 80 and the secondary end 82 of the spray nozzle 78 is open, and the outside wall 84 tapers between the primary end 80 and the secondary end 82. The second end 76 of the second hose 72 is fluidly coupled to the primary end 80 of the spray nozzle 78 to spray the disinfectant solution 36 onto the seat portion 26 of the chair 14 when the pump 62 is turned on.

In use, the cylinder 12 is attached to the bottom surface 24 of the seat portion 26 of the chair 14 and each of the attachments 48 is attached to the backrest 50 of the chair 14. The second hose 72 is drawn outwardly from the puck 54 to facilitate the spray nozzle 78 to be strategically positioned to spray the disinfectant solution 36 on the seat portion. The user 37 depresses the button 68 on the puck 54 prior to sitting in the chair 14 thereby facilitating the disinfectant solution 36 to be sprayed on and around the chair 14. In this way the chair 14 is disinfected to inhibit the chair 14 from spreading an infectious disease between multiple users. The bottle 34 in the cylinder 12 is replaced when the disinfectant solution 36 is depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A chair sanitizing assembly for spraying a sanitizing solution on a chair before a user sits in the chair, said assembly comprising:
a cylinder being configured to be attached to a chair;
a bottle being configured to contain a disinfectant solution, said bottle being insertable into said cylinder;
a first hose being fluidly coupled to said bottle wherein said first hose is configured to receive the disinfectant solution;
a puck having said first hose extending therein;
a pump being positioned within said puck, said first hose being fluidly coupled to said pump wherein said pump is configured to pump the disinfectant solution;
a button being movably integrated into said puck wherein said button is configured to be depressed by a user, said button being integrated into said pump, said pump being turned on when said button is depressed wherein said pump is configured to pump a predetermined volume of the disinfectant solution;
a second hose being fluidly coupled to said pump wherein said second hose is configured to receive the disinfectant solution when said pump is turned on, said second hose extending outwardly from said puck wherein said second hose is configured to be draped over the backrest of the chair; and
a spray nozzle being coupled to said second hose wherein said spray nozzle is configured to spray the disinfectant solution onto the chair portion of the chair thereby disinfecting the chair portion prior the user sitting on the chair portion.

2. The assembly according to claim 1, wherein said cylinder has a first end, a second end and an outer wall extending therebetween, said cylinder being hollow, said second end being open, said outer wall having a flattened portion extending between said first end and said second end wherein said flattened portion is configured to lie against a bottom surface of a seat portion of the chair.

3. The assembly according to claim 2, wherein said outer wall has a window extending therethrough, said window being elongated to extend substantially between said first end and said second end, said window being positioned on an opposite side of said outer wall with respect to said flattened portion.

4. The assembly according to claim 2, wherein said cylinder has a pair of tabs each extending away from a respective one of said first end and said second end, each of said tabs being aligned with said flattened portion, each of said tabs having a hole extending therethrough wherein said hole in each of said tabs is configured to accommodate a fastener to engage the bottom surface of the seat portion of the chair for attaching said cylinder thereto.

5. The assembly according to claim 3, wherein said bottle has an outlet being fluidly integrated into a primary end of said bottle, said bottle being insertable into said second end of said cylinder having said outlet being exposed, said bottle being comprised of a translucent material wherein said bottle is configured to facilitate the disinfectant solution to be visible therethrough such the level of the disinfectant solution is visible through said window in said outer wall of said cylinder.

6. The assembly according to claim 5, wherein said first hose has a first end and a second end, said first end of said first hose being fluidly coupled to said outlet of said bottle.

7. The assembly according to claim 6, wherein said pump has an input and an output, said second end of said first hose being fluidly coupled to said input wherein said pump is configured to pump the disinfectant solution outwardly through said output when said pump is turned on.

8. The assembly according to claim 7, further comprising a spool being positioned within said puck, said spool surrounding said pump, said spool being biased to rotate in a first direction, said spool being urgeable to rotate in a second direction, said spool being aligned with said output of said pump.

9. The assembly according to claim 8, wherein said second hose has a first end and a second end, said first end of said second hose being fluidly coupled to said output of said pump, said second hose being wrapped around said spool when said spool is biased to rotate in said first direction, said spool rotating in said second direction when said second hose is drawn outwardly from said puck.

10. The assembly according to claim 9, wherein said spray nozzle has a primary end, a secondary end and an outside wall extending therebetween, said spray nozzle being hollow, each of said primary end and said secondary end being open, said outside wall tapering between said primary end and said secondary end, said second end of said second hose being fluidly coupled to said primary end of said spray nozzle wherein said secondary end of said spray nozzle is configured to spray the disinfectant solution onto the seat portion of the chair when said pump is turned on.

11. A chair sanitizing assembly for spraying a sanitizing solution on a chair before a user sits in the chair, said assembly comprising:
a cylinder being configured to be attached to a chair, said cylinder having a first end, a second end and an outer wall extending therebetween, said cylinder being hollow, said second end being open, said outer wall having a flattened portion extending between said first end and said second end wherein said flattened portion is configured to lie against a bottom surface of a seat portion of the chair, said outer wall having a window extending therethrough, said window being elongated to extend substantially between said first end and said second end, said window being positioned on an opposite side of said outer wall with respect to said flattened portion, said cylinder having a pair of tabs each extending away from a respective one of said first end and said second end, each of said tabs being aligned with said flattened portion, each of said tabs having a hole extending therethrough wherein said hole in each of said tabs is configured to accommodate a fastener to engage the bottom surface of the seat portion of the chair for attaching said cylinder thereto;
a bottle being configured to contain a disinfectant solution, said bottle being insertable into said cylinder, said bottle having an outlet being fluidly integrated into a primary end of said bottle, said bottle being insertable into said second end of said cylinder having said outlet being exposed, said bottle being comprised of a translucent material wherein said bottle is configured to facilitate the disinfectant solution to be visible therethrough such the level of the disinfectant solution is visible through said window in said outer wall of said cylinder;
a first hose being fluidly coupled to said bottle wherein said first hose is configured to receive the disinfectant solution, said first hose having a first end and a second end, said first end of said first hose being fluidly coupled to said outlet of said bottle;
a plurality of attachments, each of said attachments having said first hose extending therethrough wherein each of said attachments is configured to engage a backrest of the chair having the first hose extending upwardly along the back rest;
a puck having said first hose extending therein, said puck having a top side, a bottom side and a perimeter side extending therethrough;
a pump being positioned within said puck, said first hose being fluidly coupled to said pump wherein said pump is configured to pump the disinfectant solution, said pump having an input and an output, said second end of said first hose being fluidly coupled to said input wherein said pump is configured to pump the disinfectant solution outwardly through said output when said pump is turned on;
a button being movably integrated into said puck wherein said button is configured to be depressed by a user, said button being integrated into said pump, said pump being turned on when said button is depressed wherein said pump is configured to pump a predetermined volume of the disinfectant solution, said button extending through said top side of said puck;
a spool being positioned within said puck, said spool surrounding said pump, said spool being biased to rotate in a first direction, said spool being urgeable to rotate in a second direction, said spool being aligned with said output of said pump;
a second hose being fluidly coupled to said pump wherein said second hose is configured to receive the disinfectant when said pump is turned on, said second hose extending outwardly from said puck wherein said second hose is configured to be draped over the backrest of the chair, said second hose having a first end and a second end, said first end of said second hose being fluidly coupled to said output of said pump, said second hose being wrapped around said spool when said spool is biased to rotate in said first direction, said spool rotating in said second direction when said second hose is drawn outwardly from said puck; and
a spray nozzle being coupled to said second hose wherein said spray nozzle is configured to spray the disinfectant solution onto the chair portion of the chair thereby disinfecting the chair portion prior the user sitting on the chair portion, said spray nozzle having a primary end, a secondary end and an outside wall extending therebetween, said spray nozzle being hollow, each of said primary end and said secondary end of said spray nozzle being open, said outside wall tapering between said primary end and said secondary end, said second end of said second hose being fluidly coupled to said primary end of said spray nozzle wherein said secondary end of said spray nozzle is configured to spray the disinfectant solution onto the seat portion of the chair when said pump is turned on.
